(19) United States
(12) Patent Application Publication
(10) Pub. No.: US 2020/0108189 A1
(43) Pub. Date: Apr. 9, 2020

(54) SURGICAL SUCTION TIP WITH INTEGRATED CLEANING MECHANISM

(71) Applicant: Peter Larson, Lake Elmo, MN (US)

(72) Inventor: Peter Larson, Lake Elmo, MN (US)

(21) Appl. No.: 16/594,128

(22) Filed: Oct. 7, 2019

Related U.S. Application Data
(60) Provisional application No. 62/742,478, filed on Oct. 8, 2018.

(51) Int. Cl. A61M 1/00

(52) U.S. Cl. CPC A61M 1/0078; A61M 1/0039

(57) ABSTRACT

A surgical tip device for coupling to a suction source. The surgical tip extending from a proximal end to a distal end and defining a cavity for the passage of suction flow through the device. The tip includes a probe movably received within the cavity and capable of manipulation from an exterior of the device to remove obstructions from both the distal end, the proximal end, and within the cavity.

### CROSS REFERENCE TO RELATED APPLICATIONS

This US patent application claims priority to U.S. Provisional Patent Application No. 62/742,478 filed 8 Oct. 2018 to the above-named inventor, the disclosure of which is considered part of the disclosure of this application and is herein incorporated by reference in its entirety.

### FEDERALLY SPONSORED RESEARCH OR DEVELOPMENT

Not Applicable

### SEQUENCE LISTING, A TABLE, OR A COMPUTER PROGRAM

Not Applicable

### FIELD OF THE INVENTION

This invention relates generally to surgical tip with an integrated cleaning mechanism within an interior space of the surgical tip to easily remove clogged debris during use.

### BACKGROUND

Several surgical procedures utilize suction as a means to aid in various medical procedures. These procedures typically utilize a suction source in communication with a suction tip to allow a physician to remove blood, bone fragments, various fluids, including those used to irrigate a surgical site, and other tissues, such as muscle and fats. In use, a physician or assistant will typically hold a proximal end of the suction tip and manipulate a distal end of the surgical tip within a surgical cavity for the removal of the unwanted materials during a given procedure.

A typical surgical tip utilizes a generally rigid tubular body forming a cavity for the flow of suction from the suction source. This tip may be constructed out of plastic, metal, or other similar material. Often during use, the cavity of the surgical tip can become clogged due to the size and volume of tissues and fragments. When a clog occurs, suction is impacted and removal of the clog is required to maintain optimal suction. To clear the clog, often a probe or other similar tool is inserted into the cavity at the distal end to try and break up the clog and force the material out of the tip and towards the suction source. Still further, some clogs may require a changing of tips. Accordingly, these repeated clogs result in unnecessary delays and costs for a given procedure.

Therefore, there is a need within the market for an improved surgical tip with an ability to easily remove clogs during use. Preferably, this tip is easy to use, durable, and effective for most clogs.

### BRIEF SUMMARY OF THE INVENTION

In one aspect, this disclosure is related to a surgical tip configured for the removal of internal clogs within the surgical tip without the use of an additional tool.

In another aspect, this disclosure is related to a surgical tip device having a mechanism placed within an interior space to easily remove clogs during use of the device without the use of any external intervention.

In another aspect, this disclosure is related to a surgical tip having a cavity in communication with a suction source including an internal probe positioned on a slide and movable within the cavity for clearing a blockage.

The device of the present disclosure provides a surgical aspiration tip having a proximal end coupled to a suction source opposite a distal end for manipulation within a surgical site. The surgical tip generally forming a cavity along the device length between the proximal end and the distal end and in pneumatic and fluid communication with the suction source. The cavity generally a hollow tubular structure having diameter sufficiently sized for the removal of fluids, tissues, and debris from a surgical site. A movable probe is received within the cavity for manipulation by a user of the device, generally and preferably through a sliding “back-and-forth” motion. Accordingly, the probe is coupled to a slide member configured for manipulation by a user of the device external to the interior cavity.

### BRIEF DESCRIPTION OF THE DRAWINGS

The accompanying drawings are included to provide a further understanding of the present invention and are incorporated in and constitute a part of this specification. The drawings illustrate exemplary embodiments of the present invention and together with the description serve to further explain the principles of the invention. Other aspects of the invention and the advantages of the invention will be better appreciated as they become better understood by reference to the Detailed Description when considered in conjunction with accompanying drawings, and wherein:

FIG. 1 is an isometric view of the device with probe extended, according to the present disclosure;

FIG. 2 is a side view of the device, according to the present disclosure;

FIG. 3 is a top side view of the device, according to the present disclosure;

FIG. 4 is a bottom view of the device, according to the present disclosure;

FIG. 5 is an end view of the device, according to the present disclosure; and

FIG. 6 an isometric view of the device, according to the present disclosure.

### DETAILED DESCRIPTION OF THE INVENTION

The following detailed description includes references to the accompanying drawing, which forms a part of the detailed description. The drawing shows, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful, and is not intended to exclude other embodiments from the scope of the invention.

As used herein, the terms “front,” “back,” “rear,” “upper,” “lower,” “right,” and “left” in this description are merely used to identify the various elements as they are oriented in the FIGS, with “front,” “back,” and “rear” being relative to the apparatus. These terms are not meant to limit the elements that they describe, as the various elements may be oriented differently in various applications.

As used herein, the term “coupled” means the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

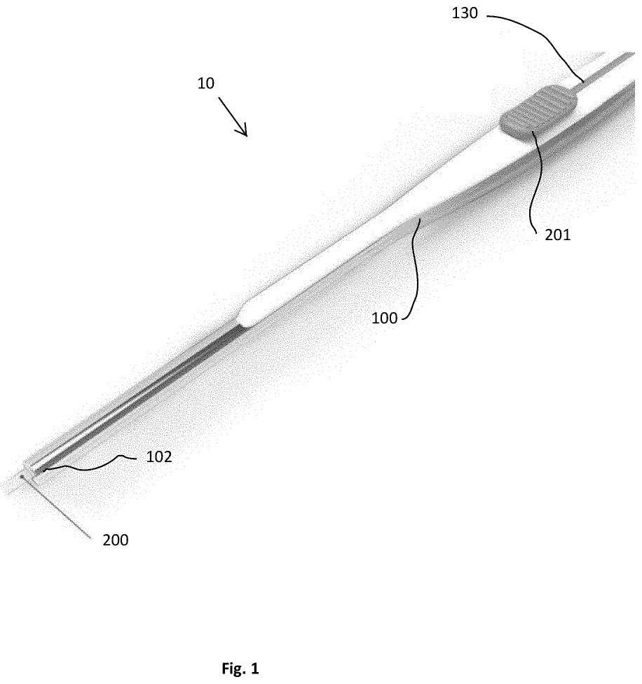

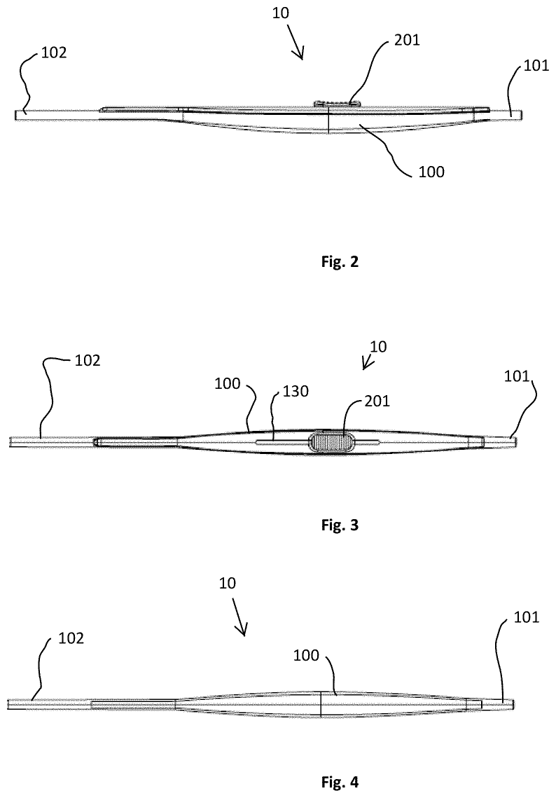

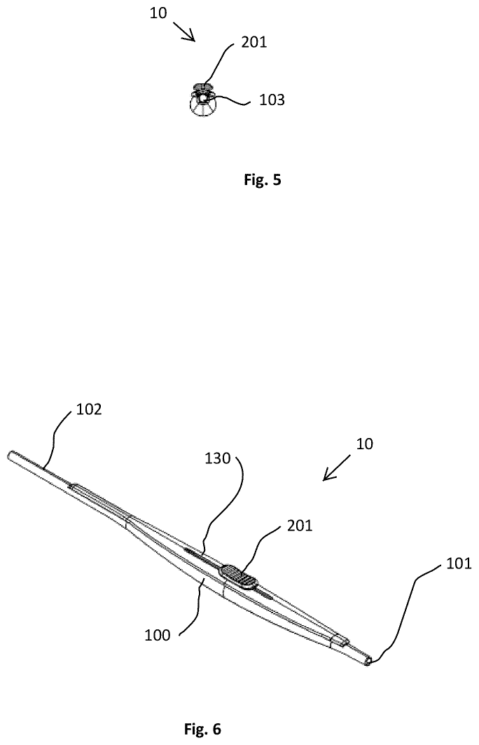

Referring now to FIGS. 1-6 of the surgical tip with integrated cleaning mechanism generally referred to as device 10. The device 10 is configured for attachment to a suction source and associated couplings for functioning as surgical aspirator tip for the removal of fragments, fluids, and tissues from a surgical site and associated cavities. The device 10 is comprised of a housing 100 having a proximal end 101 and a distal end 102 opposite the proximal end 101. The proximal end 101 configured for a coupling with a standard hose or tube delivering suction for the aspiration of a surgical site through use of the device 10 and directed towards the distal end 102 for placement into the surgical site or cavity.

The housing 100 having a shape defining an exterior and an interior space. The interior space further defining a cavity 103 extending from the proximal end 101 to the distal end 102. The cavity 103 being hollow and tubular allowing for the flow of materials, tissues, fragments, and fluids to the suction source for aspiration of the surgical site.

A probe 200 is movably positioned within the cavity 103 at a position coplanar with and extending parallel to the cavity 103 at least from a first position adjacent the proximal end 101 to a second position adjacent the distal end 102 of the housing 100, wherein the probe 200 is configured to mechanically move between the first position and the second position to dislodge and remove stuck fragments and tissue from within the cavity 103 at the proximal end 101 and the distal end 102. In the preferred embodiment of the present disclosure, the probe 200 has a length generally corresponding to a distance between the proximal end 101 and the distal end 102 within the cavity 103 of the device 10.

The probe 200 is positioned within the cavity 103 at a central position when compared to a diameter of the proximal end 101 and the distal end 102, wherein the probe 200 is generally configured to remove stuck fragments from all positions within the diameter of the cavity 103 when manipulated by a user of the device 10. Accordingly, the probe 200 is coupled to a sliding mechanism configured for manipulation of the probe 200 from a position at the exterior of the housing 100 by a user of the device 10.

The sliding mechanism of the probe 200 is comprised of a tab 201 coupled to a generally middle portion of the length the probe 200 and movable within a channel 130 between the proximal end 101 and distal end 102 of the housing, wherein movement of the tab 201 is translated to the probe 200. The channel 130 is sealed with respect to the cavity 103, wherein the channel 130 does not affect the suction of the device 10. Accordingly, the channel 130 generally includes a resilient member coupled to the tab 201 or the channel 130 to generally seal a channel 130 aperture with respect to the cavity 103 to maintain suction during device 10 use.

As is seen in FIG. 1, this sliding mechanism of the probe 200 generally allows for movement of the probe 200 to a position external to the housing 100 at the proximal end 101 and the distal end 102, wherein the probe 200 first position extends out of the cavity 103 of the housing 100 proximal end 101 and the probe 200 second position extends out of the cavity 103 of the housing 100 distal end 102 to ensure adequate cleaning of the cavity 103 during manipulation. Accordingly, a user of the device 10 will easily manipulate the tab 201 by sliding the tab 201 in a back and forth motion to clear the cavity 103 of any obstructions infringing the suction from the suction source.

Although it is preferred that the probe 200 has movement between both the proximal end 101 and the distal end 102 to clear a blockage within the cavity 103 at both ends, the probe 200 can alternately be configured for movement to only move towards the proximal end 101 or the distal end 102. Accordingly, the sliding mechanism of the probe 200 can be configured to only clear a blockage at either the proximal end 101 or the distal end 102 or both the proximal end 101 and the distal end 102.

Further, it is preferred that the housing 100 and overall shape of the device 10 is configured with a shape that is generally considered ergonomic and comfortable. Accordingly, the housing 100 shape may have various curves and projections for receipt comfortably within the palm and hand of a user. Additionally, the tab 201 having a shape for easy manipulation by a user. It is expected that the device 10 housing 100 will be comfortably received within the fingers and grasped within a hand of the user with the tab 201 aligned for manipulation and sliding by a thumb of the user, wherein the user can quickly and efficiently use their thumb in a sliding motion to move the probe 200 within the cavity 103 to remove an obstruction.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) but that the invention will include all embodiments falling with the scope of the appended claims.

What is claimed is:

1. A suction tip, the suction tip configured for coupling to a suction source for manipulation within a surgical site for the removal of objects, fragments, tissues, fluids, debris, and other similar materials, for aspiration of a surgical site, the suction tip comprising:
a housing, the housing comprising:
a proximal end, the proximal end configured for coupling to the suction source;
a distal end, the distal end opposite the proximal end; and
a cavity, the cavity extending a length defined as a distance between the proximal end and the distal end; and
a probe, the probe received within the cavity and movable between a first position adjacent to the proximal end.

2. A suction tip as in claim, wherein the prove is movable between the first position and a second position adjacent to the distal end.

3. A suction tip as in claim 2, wherein the housing includes a channel, the channel in communication with the cavity, the channel sized to receive a tab coupled to the probe, wherein movement of the tab is translated to the probe.

4. A suction tip as in claim 3, wherein the channel is sealed with respect to the cavity.

5. A suction tip as in claim 2, wherein the probe has a length, the length corresponding to the distance between the proximal end and the distal end of the housing.

6. A suction tip as in claim 2, wherein the housing has a shape that is ergonomic with respect to grasping by a user.

7. A suction tip as in claim 2, wherein the probe is centrally received within a diameter of the cavity.

8. A suction tip, the suction tip configured for coupling to a suction source for manipulation within a surgical site for the removal of objects, fragments, tissues, fluids, debris, and other similar materials, for the aspiration of a surgical site, the suction tip comprising:
a housing, the housing comprising:
a proximal end, the proximal end configured for coupling to the suction source;
a distal end, the distal end opposite the proximal end; and
a cavity, the cavity having a diameter extending a length defined as a distance between the proximal end and the distal end; and
a probe, the probe centrally received within the diameter of the cavity and movable between a first position adjacent to the proximal end and a second position adjacent to the distal end.

9. A suction tip as in claim 8, wherein the housing includes a channel, the channel in communication with the cavity, the channel receiving a tab coupled to the probe, wherein movement of the tab is translated to the probe.

10. A suction tip as in claim 8, wherein the channel is sealed with respect to the cavity.

11. A suction tip as in claim 8, wherein the housing has a shape that is ergonomic with respect to grasping by a user.

12. A suction tip as in claim 8, wherein the probe is positioned coplanar within the cavity.

13. A suction tip as in claim 8, wherein the tab is coupled to a middle of the length of the probe.

14. A suction tip, the suction tip configured for coupling to a suction source for manipulation within a surgical site for the removal of objects, fragments, tissues, fluids, debris, and other similar materials, for the aspiration of a surgical site, the suction tip comprising:
a housing, the housing comprising:
a proximal end, the proximal end configured for coupling to the suction source;
a distal end, the distal end opposite the proximal end;
a cavity, the cavity having a diameter extending a length defined as a distance between the proximal end and the distal end; and
a channel, the channel an aperture through the housing in communication with the cavity; and
a probe, the probe centrally received within the diameter of the cavity and movable between a first position adjacent to the proximal end and a second position adjacent to the distal end.

15. A suction tip as in claim 14, wherein the probe includes a tab.

16. A suction tip as in claim 15, wherein the tab is received within the channel for translating movement of the tab to the probe.

17. A suction tip as in claim 14, wherein the channel is sealed with respect to the cavity.

18. A suction tip as in claim 14, wherein the housing has a shape that is ergonomic with respect to grasping by a user.

19. A suction tip as in claim 14, wherein the probe is positioned coplanar within the cavity.

20. A suction tip as in claim 15, wherein the tab is coupled to a middle of the length of the probe.
